(19) United States
(12) Patent Application Publication
(10) Pub. No.: US 2024/0408377 A1
(43) Pub. Date: Dec. 12, 2024

(54) PERISTALTIC PUMP AND ROCKER USED THEREWITH

(71) Applicant: B. Braun Avitum AG, Melsungen (DE)

(72) Inventors: Marco Jakobi, Uder (DE); Oliver Schaefer, Neuenstein (DE); Bruno Stenzel, Hann. Muenden (DE)

(21) Appl. No.: 18/735,821

(22) Filed: Jun. 6, 2024

(30) Foreign Application Priority Data
Jun. 7, 2023 (DE) 10 2023 115 068.5

(51) Int. Cl. A61M 60/279; A61M 60/113; A61M 60/37; A61M 60/835

(52) U.S. Cl. CPC A61M 60/279; A61M 60/113; A61M 60/37; A61M 60/835; A61M 2207/00

(57) ABSTRACT

A peristaltic pump, specifically for pumping fluid in an extracorporeal blood treatment apparatus, includes a pump housing that accommodates a rotor rotatable about a rotor axis and a squeeze element. The pump housing includes a support surface extending in a curved shape around the rotor axis and being radially spaced from the rotor. The support surface can support a tube segment that inserted radially between the rotor and support surface. The rotor supports the squeeze element via a rocker mounted on the rotor in a pivotable and cushioned manner. The rocker supports an insertion aid by which the tube segment can be pressed into the pump bed, and tube guide elements by which the tube segment is centrally alignable to the squeeze element. The rocker is made from high-strength stainless steel with high chemical resistance and manufactured by powder injection molding, such as metal injection molding.

### CROSS-REFERENCE TO RELATED APPLICATION

This application claims priority under 35 U.S.C. § 119 to German Application No. 10 2023 115 068.5, filed on Jun. 7, 2023, the content of which is incorporated by reference herein in its entirety.

### FIELD

The present disclosure relates to a tube roller pump or peristaltic pump, i.e. a positive-displacement pump, in which the fluid to be pumped is pressed through a tube by external mechanical deformation of the latter, as well as to a rocker tailored for said peristaltic pump. Pumps of this type are frequently used to pump fluid, specifically blood, in an apparatus for extracorporeal blood treatment, particularly in a dialysis machine. The fluid is pumped by means of the peristaltic pump from a low-pressure side to a high-pressure side, wherein an elastically deformable fluid line disposed between the low-pressure side and the high-pressure side in the form of a tube segment referred to as pump segment is deformed, specifically squeezed, between a support surface of a pump bed and a rotor rotating relative to said support surface and having at least two squeeze elements.

### BACKGROUND

As a rule, in a peristaltic pump, as it is known, for example, from EP 1 749 549 B1 and is schematically shown in FIGS. 8 and 9, at least two squeeze elements 320 offset against each other in the circumferential direction in the form of spring-mounted pinch or pressure rollers are provided on the rotor rotatably supported in the pump housing. The support surface 330 is formed by a circle segment area which extends over a sufficiently large central angle of more than 180°. The pressure rollers are rotatably supported by means of a roller axle 321 and are located on swing arms or rockers 341 which are articulated on the rotor 310 by means of a hinge pin 338 with an axis A338 and are subjected to a radially outwardly directed force FR (see FIG. 8) by a compression spring 336.

In order to take the peristaltic pump into operation, it is necessary to pass a tube segment not shown in detail in FIG. 8, viz. the pump segment, from a low-pressure side connection PN in arc or curved shape above the rotor 310 following the support surface 330 to a high-pressure side connection PP and then to thread it into the pump bed 307 by manually rotating the rotor 310 in the direction of the arrows D. The known peristaltic pump includes, as a threading or insertion aid to assist manual threading, two guide projections supported by the rotor 310 and being in the form of hooks 360 which are interposed between the two pinch or pressure rollers 320. Differently from concepts with automatic threading of the pump segment in which the rotor is automatically aligned to an optimum threading position, in the case of the peristaltic pump known from EP 1 749 549 B1, the user must ensure that the tube is inserted upstream of the hook 360 to prevent jamming. For this purpose, the rotor 310 must be aligned manually in a position as shown in FIG. 8. For guiding the tube segment already caught by the hooks 360 and for aligning the tube segment centrally to the squeeze element, guide pins 370 additionally inserted in the rotor and acting as tube guide elements are used, which guide pins 370 trail the hooks 360.

In order to simplify the threading operation, it would be advantageous to integrate the insertion aid and/or the tube guide elements into the rocker. However, this renders the shape of the rocker even more complex, which results in an even more complicated manufacture of the rocker.

Therefore, known rockers of rotors in blood pumps are either plastic injection molded parts or mechanically manufactured parts made from plastic or metal.

Since rockers of this type require high dimensional stability and fatigue strength due to the bending forces to be cyclically absorbed, applicant already attempted to manufacture rockers with an integrated tube guide made from aluminum and to produce the contour from the solid by a milling method. Even if the tube guide elements are designed as separate pins, in this case plenty of material has to be removed usually by chipping so that the use of raw material compared to the finished part becomes very high. It is another problem that the rocker has to be crimped with the roller axle for fastening the squeeze element and, resp., the pressure roller. Due to the low strength of the aluminum, slight deformations may occur when the roller axles are crimped. In order to avoid rejects, therefore an accurate process monitoring is required. In addition, the milled aluminum rockers still have to be anodized so as to ensure the chemical resistance. Since anodizing affects the accuracy of the fits in the boreholes, the boreholes have to be plugged before anodizing. The steps of anodizing and plugging constitute additional expenditure. When high-strength materials are used, the mechanical machining becomes even more complicated and more cost-intensive.

### SUMMARY

The object underlying the present disclosure is to provide a generic peristaltic pump which excels by simplified manufacture while the functionality and the service life are increased.

According to the innovation, the rocker of the rotor is made of high-strength stainless steel having excellent chemical properties, the rocker being manufactured by the MIM (metal injection molding) method. Said manufacture allows to integrate additional functions such as a threading aid and a tube segment guide, while at the same time the raw material used and the manufacturing costs are considerably reduced as compared to metal-cutting manufacturing. Accordingly, the shaping MIM method helps minimize the mechanical finishing. In addition, delicate contours (e.g. reinforcing ribs) can be realized by the MIM method, allowing the mass of the component to be further reduced while maintaining the strength. It is difficult or even impossible to produce said delicate contours by milling. Thanks to the high surface quality that can be achieved by the MIM method, in the area of the surfaces getting into contact with the tube segment no specific reworking is required, which can be additionally exploited to reduce the manufacturing costs.

Tests have shown that those materials such as an austenitic heat-resistant chromium-nickel steel, as it is known e.g. under the designation 1.4841 (X15CrNiSi25-21), can be easily produced with sufficiently high densities by the MIM method, and that the component parts manufactured therefrom have optimum properties for use in peristaltic pumps. The rocker manufactured in this way is chemically resistant to various fluids and, with a minimum use of material of only few cm³, has such high strength that, on the one hand, the pressure of the rollers in the pumping operation of the various fluids is ensured by a delicate design and, on the other hand, crimping of the roller axles is guaranteed without the rockers being deformed. This results in the additional advantage that, due to the chemical properties of the material, an additional surface treatment such as varnishing or anodizing is not necessary.

The selection of the material and the manufacturing method according to the innovation offers the option of transferring additional functions to the rocker in a compact design, such as integrating the components for guiding the tube segment into the rocker in such a way that they are located close to the holder of the squeeze element and the pressure roller, respectively. In this way, by means of a bridging member framing the pressure roller, the rocker can form a bearing position on both sides for the pressure roller, allowing the forces from the pressure roller to be transferred safely and with little deformation to the rocker.

In further embodiments, configurations of the rocker are determined by which the operation of the pump, in particular the handling when threading in and/or out and when centrically guiding the tube segment received in the pump, viz. the pump segment, centrally to the pressure rollers, is facilitated. At the same time, this results in an increasingly more complex shape of the rocker. While the resulting free spaces or clearances of the rocker are created during the metal-cutting machining of a blank by material removal, in the configuration of the rocker according to the innovation only the material required is injected into the forming tool. The finished component part only has a volume of about 4 cm³, for example, whereas for a component part milled from the solid a semi-finished part having a volume of at least 40 cm³ would be required. Thus, raw materials can be saved to a considerable extent by the MIM method. Since even undercut ribs can be easily produced by the manufacture of the rocker according to the innovation, the mass of the rocker can be reduced while maintaining the same strength.

Apart from advantages in terms of manufacture, the configuration of the rocker according to the innovation also results in advantages during operation of the peristaltic pump. As both the insertion aid and the tube guide elements are integrated in the rocker adjacent to the squeeze element in the direction of rotation of the rotor, the number of parts and, consequently, also the manufacturing and assembly costs of the pump are reduced, because only the mounting of the rocker is necessary. As the insertion aids are located adjacent to and thus directly upstream of the associated squeeze element, it is possible for the user to manually insert the pump segment without having to comply with a specific position of the rotor.

Advantageously, the rocker is arranged on the rotor by means of a swivel bearing which is arranged offset against the squeeze element in the direction of rotation of the rotor, the insertion aid and the tube guide elements being positioned on the swivel bearing. This configuration helps maintain a particularly compact design of the rocker with the integrated insertion aid and the tube guide.

It has shown in tests that, due to the arrangement of the insertion aid on the swivel bearing of the rocker according to the innovation, it is sufficient for smoothly and reliably threading in the tube segment to design the insertion aid as a simple pin-like conduit or guide body which extends from the swivel bearing substantially in the radial direction away from the rotor axle in the direction of the support surface and which, as seen in cross-section, substantially has a rounded wedge shape facing the direction of rotation of the rotor which is inclined relative to the pump bed at an acute angle, preferably between 3° and 50°.

It is further advantageous for the guide body to include two functional surfaces the outer functional surface of which leading in the direction of rotation of the rotor and being remote from a central plane of the rocker forms a rounded wedge surface and the inner functional surface of which facing the central plane is formed by a cylindrical segment surface preferably steplessly adjoining the wedge surface. The rounded wedge surface presses the tube segment to be threaded in gently downwards into the pump bed while the rotor is manually rotated, the rounded geometry preventing the tube segment from being damaged. The cylindrical segment surface adjoining the wedge surface can advantageously be used to guide the tube segment centrally to the squeeze elements, when the peristaltic pump is operated. In doing so, care is taken, of course, that the quality of the surfaces of the guide body getting into contact with the tube segment is such that the tube segment is protected in the best possible way. It has turned out, that said surface quality, e.g. a sufficiently small roughness, can be achieved by the MIM manufacturing method without any further treatment of functional surfaces.

The MIM method also ensures the dimensional accuracy of the pin-like guide body and the allocation of positions to the bearing axes by taking advantage of the linear shrinkage during the sintering process and by precisely calculating the shrinkage in advance by test sintering processes.

The cross-section of the guide body may take diverse shapes as long as the wedge surface and the cylindrical segment surface are formed on the side facing the tube segment in accordance with the application. If, however, the cross-section of the guide body has a central axis, this results in a simplified manufacture in particular in case that the guide body is mounted on the rocker as a separate component. For the alternative case that the rocker and the guide body are formed integrally, it is of additional advantage when the guide body is supported on the rocker additionally by means of a brace following the central axis. This configuration allows to design the guide body in an even more delicate and still sufficiently stable manner.

If the guide body is arranged, and preferably integrally mounted, on a swivel bearing eye of the rocker, the radial supporting forces and bending forces applied to the guide body during threading can be properly transferred to the rocker, as the swivel bearing eye is stably supported by the swivel axle and, consequently, is subjected to minimum deformations only.

A further advantageous configuration of the peristaltic pump consists in equipping the rocker at its end section remote from the swivel bearing eye with an abutment surface for a compression spring and in arranging, such as rotatably supporting, the squeeze element between the swivel bearing eye and the abutment surface. The MIM manufacturing method allows to optimally position the abutment surface without rendering the manufacture of the rocker more complex. In this way, a compact shape is imparted to the swing arm so that sufficient clearance for the design of further functional surfaces, such as guide surfaces for the tube segment, is retained at the circumference of the rotor. The individual sections of the rocker can be reinforced at will where additional strength is important, for example at the zones forming the swivel bearing eye or the pivot shaft for the pressure roller, wherein the additional material expenditure required to this end remains minimized.

Preferably, a further second guide body which is designed and aligned symmetrically to the first guide body relative to a horizontal plane in parallel to the pump bed is associated with the first guide body forming the above-described insertion aid. Thus, the cylinder segment surfaces facing each other of the two mirror-inverted guide bodies form the functional surfaces of the tube guide elements. The integration of the insertion aid and the tube guide elements into the rocker is ensured with a further reduced manufacturing effort. As the guide bodies are arranged and aligned to be mirror-inverted, they guide the tube segment, when the peristaltic pump is operated, in a funnel-shaped and gentle manner into the center of the respective squeeze element, which has a positive effect on the pumping accuracy of the peristaltic pump.

The injection mold for the manufacture of the rocker is simplified, if the horizontal plane to which the two guide bodies are symmetrical is simultaneously a plane of symmetry of the rocker.

Basically, the selection of the material for manufacturing the rocker allows to leave the surfaces of the sintered rocker unmachined. However, in selected cases, the tube segment can be additionally protected from wear, if the insertion aid and, preferably, also the tube guide elements, are coated with a material that has abrasion-minimizing sliding properties.

The insertion aid and the tube guiding geometry are integrated in the rocker according to an advantageous configuration. This results in a reduction of the manufacturing and assembly costs. The tube guiding geometry has two zones. The zone marked by a dot-dash line in FIG. 4 at the upper guide body or cone is a rounded inclined surface which presses the tube downwards during threading. The rounded geometry prevents the tube from being damaged. The round surfaces marked by broken lines in FIG. 4 at the upper and lower guide bodies or cones guide the tube during operation so that the pump segment is guided centrally over the pressure rollers. In the area of the tube guide, the quality of the surfaces is selected such that the tube is not damaged. The surface quality is produced without any specific reworking. By arranging the insertion aid directly upstream of the pressure rollers, the user does not have to comply with any specific position of the rotor during manual threading.

### BRIEF DESCRIPTION OF THE DRAWINGS

In the following, embodiments of the present disclosure are illustrated in detail by means of schematic drawings, wherein:

FIG. 1 shows a perspective representation of an exemplary embodiment of a peristaltic pump, as it can be applied in connection with an apparatus for extracorporeal blood treatment;

FIG. 2 shows a perspective view of a rotor used in the peristaltic pump according to FIG. 1;

FIG. 3 shows a perspective view of the rotor base body of the peristaltic pump with a mounted rocker;

FIG. 4 shows, in greatly enlarged scale, the lateral view of the rocker used in the peristaltic pump according to FIGS. 1 and 2, when viewed along the arrow “IV” in FIG. 2;

FIG. 5 shows a perspective view of the rocker;

FIG. 6 shows a view of the rocker corresponding to FIG. 5 having a schematically indicated material volume that would be required for a manufacturing variant;

FIGS. 7A and 7B show perspective views of rockers manufactured by machining;

FIG. 8 shows a top view of a known peristaltic pump; and

FIG. 9 shows a perspective view of the rotor used in the peristaltic pump according to FIG. 8.

### DETAILED DESCRIPTION

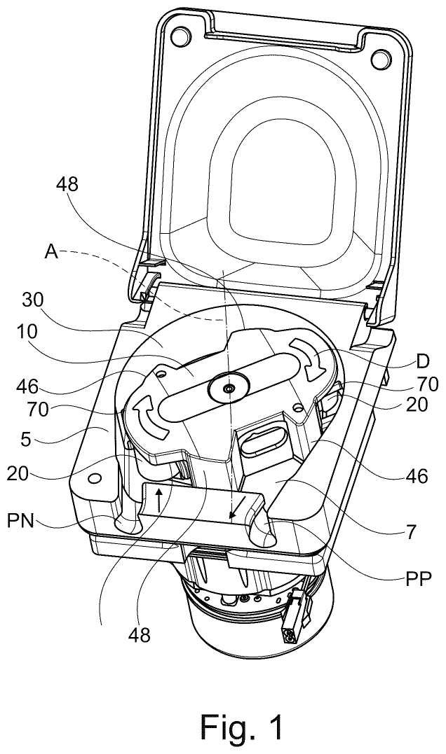

FIG. 1 illustrates a peristaltic pump as it is utilized in dialysis machines, for example. In those cases, the function of the peristaltic pump is to convey or pump a defined volume of a fluid, such as blood or dialysis fluid, by deforming and pinching the elastically deformable fluid line. The peristaltic pump for pumping blood usually pumps from a negative pressure side PN (low-pressure terminal section or connection) to a positive pressure side PP (high-pressure terminal section or connection).

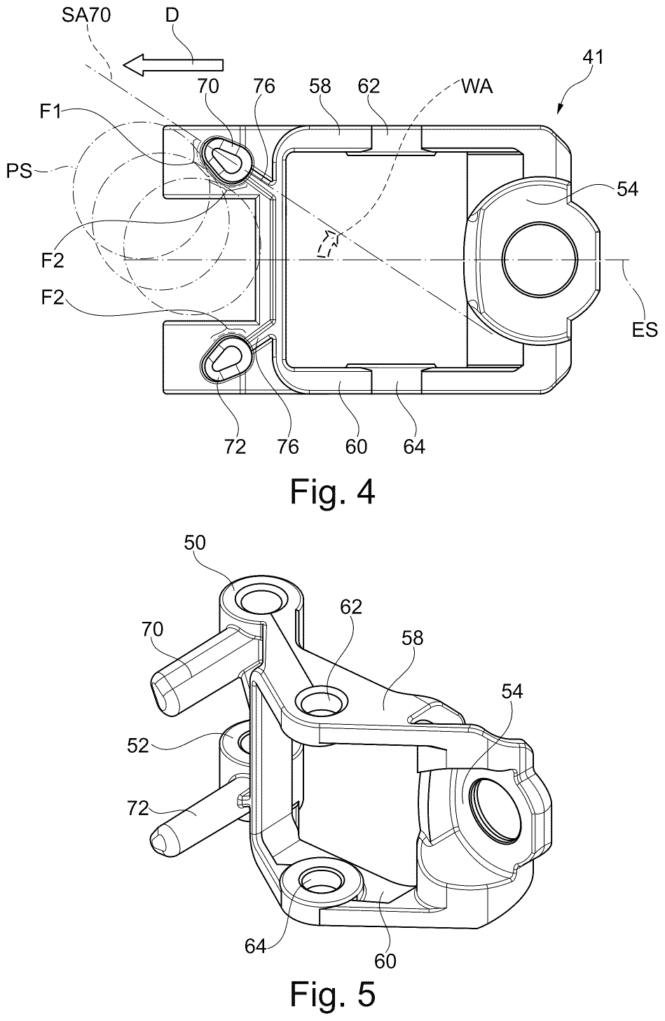

As illustrated in FIG. 1, the peristaltic pump includes a rotor or pump housing 5—shown unfolded in FIG. 1—in which a rotor 10 rotatable about a rotor axis A and having at least two squeeze elements 20, pressure rollers in this case, offset in the circumferential direction, such as diametrically, against each other, is housed. The pump housing 5 comprises a pump bed 7 which extends in arc or curved shape around the rotor axis A and includes a support surface 30 radially spaced apart from the rotor 10. Said support surface 30 is arranged to radially support a pump segment PS (not shown in the Figures and indicated by a dot-dash line in FIG. 4 only) which can be introduced radially between itself and the rotor 20. The squeeze elements 20 are supported and cushioned on a rocker not designated in detail in FIG. 1 which, in turn, is pivotably fastened on the rotor 10 via a hinge pin 38 (seen in FIG. 3). The squeeze elements 20 are rotatably mounted on a rocker 41 articulated in a spring-mounted manner on the rotor base body 40.

The tube segment inserted in the peristaltic pump shall hereinafter be referred to as pump segment. The tube segment—not shown in FIG. 1—does not bear on a pump bed provided with reference numeral 7, but it is positioned centrally to the squeeze elements by tube guide elements described in detail below. The support surface 30 is usually formed by a cylindrical surface, but it can also be recessed in trough shape. The direction of rotation of the rotor 10 when fluid is pumped is denoted with the arrow D.

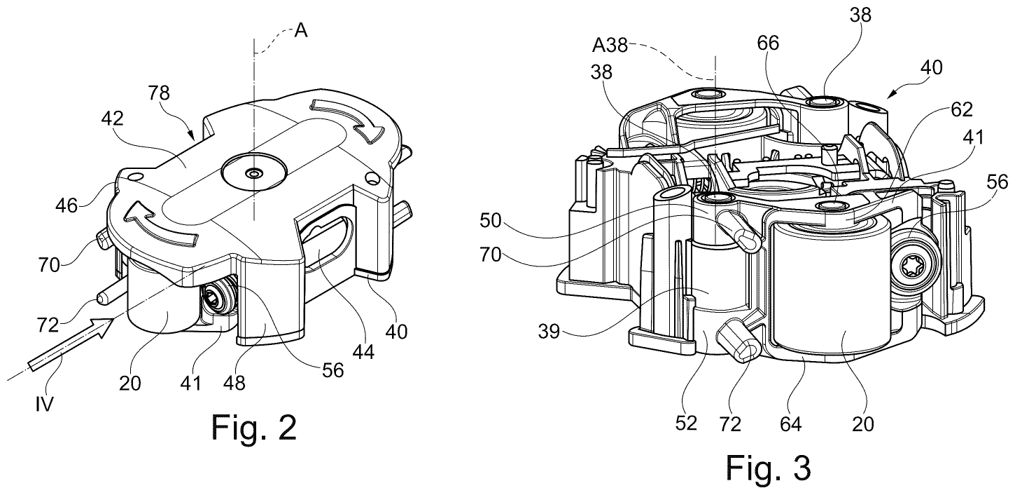

FIG. 2 illustrates details of the rotor 10. It has a rotor base body 40 shown in perspective in FIG. 3 and a rotor cover 42. The rotor 10 can be removed from a drive shaft not shown in detail by a push button 44 operable from the side.

The rotor cover 42 co-rotating with the rotor 10 supports guide surfaces 46, 48 in the area of the squeeze elements 20, the one guide surface 46 leading the squeeze element 20 and the other guide surface 46 trailing the squeeze element 20. Accordingly, the guide surfaces 46, 48 are arranged and configured on both sides of the squeeze element 20 adjacent in the circumferential direction so that each of them forms a circle segment-type guide passage with the support surface 30 in which guide passage a pump segment inserted into the pump bed 7 is radially caught with a predetermined clearance fit. When the peristaltic pump is operated, the pump segment can be fixed to the pump housing 5 by means of fitting bodies not shown in detail which can be secured against displacement in corresponding terminals or connections of the pump housing 5.

Before taking the peristaltic pump into operation, a tube segment which during operation of the pump becomes the pump segment has to be introduced or inserted into the pump bed 7 and has to be positioned centrally with respect to the squeeze elements 20.

For this purpose, the pump segment (not shown) is initially placed into the pump housing so that it extends from the low-pressure side connection PN in curved shape above the rotor 10 following the support surface 30 to a high-pressure side connection PP. The pump segment PS (indicated in FIG. 4 by a dot-dash line) in this phase is still located completely above the rotor 10, and more precisely above the rotor cover 42. In order to thread the pump segment into the pump bed 7 by manually rotating the rotor 10 in the direction of the arrow D, while keeping the stresses on the pump segment as low as possible and the handling as simple as possible, the rotor 10 supports an insertion aid by which the tube or pump segment, resp., while being threaded in, can be pressed into the pump bed 7, and tube guide elements by which the tube or pump segment, resp., can be aligned centrally to the squeeze elements 20.

The insertion aid and the tube guide elements are integrated, according to the innovation, in the rocker 41, which will be described in detail hereinafter with reference to the FIGS. 3 to 5.

The support of the insertion aid and the tube guide elements in the innovation is the rocker 41 pivotably attached to the rotor base body 40. More precisely, the insertion aid and the tube guide elements are integrated in the rocker 41 as follows:

As illustrated in FIG. 3 showing the rotor base body 40 from which the rotor cover 42 is removed, the rocker 41 with the swivel axis A38 is pivotably fixed to the hinge pin 38 by means of two swivel bearing eyes 50, 52. At its end section remote from the swing bearing eyes 50, 52, the rocker 41 has—as can be seen from FIGS. 3 to 6—an abutment surface 54 for a compression spring 56 by which a pressure directed away from the rotor base body 40 is applied to the rocker 41. As can be seen best from the representation according to FIG. 5, between the swivel bearing eyes 50, 52 and the abutment surface 54 the rocker 41 is in the form of a slightly convexly curved bracket having two bracket arms 58, 60 at a parallel distance which in turn form bearing sections or positions 62, 64 for a pivot shaft 66 (see FIG. 3) of the pressure roller 20. The rocker 41 has a plane of symmetry ES relative to which also the pressure roller 20 is positioned centrically in this way.

Conduit or guide bodies 70, 72 formed integrally with the swivel bearing, more precisely with the bearing eyes 50, 52, and thus integrated in the rocker 41 as closely as possible to the pressure rollers 20 are used as insertion aid and as guide elements for the pump segment PS, wherein the guide bodies extend from the swivel bearing, viz. from the axis A38 of the hinge pin 38 substantially in the radial direction from the rotor axis A, viz. from the rotor base body 40, so far that they come as close as possible to the support surface 30 when the rotor 10 rotates—as is clear from FIG. 1 in which only an upper guide body 70 is visible, however-.

The upper guide body 70 forms the insertion aid, the function of which will be described in more detail below, and both guide bodies 70, 72 together act as tube guide elements by which the pump segment is aligned centrally to the squeeze elements and pressure rollers 20, respectively.

At least the upper guide body 70 forming the insertion aid but in the shown embodiment both guide bodies 70, 72 have, as viewed in cross-section and in the radial view according to FIG. 3, a substantially rounded wedge shape pointing in or facing the direction of rotation D of the rotor which is inclined at an acute angle WA (see FIG. 4) relative to the pump bed 7 and, thus, to the plane of symmetry ES of the rocker 41 parallel thereto.

At least the upper guide body 70 has two functional surfaces F1 and F2 the outer functional surface F1 of which leading in the direction of rotation D and remote from the plane of symmetry ES, which is marked by the dot-dash line in FIG. 4, forms a rounded wedge surface, and the inner functional surface F2 of which facing the plane of symmetry ES, which is marked by a broken line in FIG. 4, is formed by a cylindrical segment surface preferably steplessly adjoining the wedge surface. The lower guide body 72 forms at least the inner functional surface F2. The functional surface F1 acts as an insertion aid when the pump segment PS is inserted, and the functional surfaces F2 facing each other act as tube guide elements to keep the pump segment centrally to the squeeze elements, i.e. the pressure rollers 20, when the peristaltic pump is operated.

In the shown embodiment, the cross-section of each of the guide bodies 70, 72 has a central axis or axis of symmetry, wherein only the axis of symmetry SA70 of the guide body 70 is plotted in FIG. 4. FIG. 4 further reveals that the guide bodies 70, 72 are additionally supported on the rocker 41 by means of a crosspiece or brace 76 following the axis of symmetry SA70.

Summing up, the upper guide body 70 with the outer functional surface F1 thus forms the insertion aid for the pump segment PS, while both guide bodies 70, 72 together with their functional surfaces F2 are used as tube guide elements.

This configuration results in the following mode of operation:

Starting from any position of rotation of the rotor 10 above which the inserted pump segment indicated in FIG. 4 by a dot-dash line and being denoted with the reference symbol PS extends from the connection point PN to the connection point PP following the support surface 30, the rotor 10 can be rotated manually by the user gripping the rotor 10 in the clearance 78 (see FIG. 2) between the guide surfaces 46, 48. The guide body 70 does not yet contact the pump segment PS in this phase.

If the rotor is turned further, the outer functional surface F1 of the guide body 70 slides onto the pump segment PS—as indicated in FIG. 4 in sequential positions—and when the rotor 10 is turned further, the functional surface F1 of the guide body 70 presses the pump segment PS more and more downwards in the direction of the pump bed so that the pump segment being adjacent to the support surface 30 is finally caught by the functional surfaces F2 centrally to the plane of symmetry ES. When the support surface 30 is passed over or swept, this function will equally continue so that finally the whole pump segment is located centrically relative to the pressure rollers 7 in the pump bed 7.

When the other guide body (not denoted in the figures), which is offset by 180° against the guide body 70 and was not involved in the threading operation, impinges on the already threaded pump segment PS, if the rotor is turned further, the guide bodies 70 and 72 in this phase approach the pump segment in such a way that they guide the pump segment funnel-like between the functional surfaces F2 and thus align it centrically relative to the pressure rollers 20. This mode of action results each time when, during operation of the peristaltic pump, a pressure roller comes into operation, allowing the displacement volume of the peristaltic pump to be kept constant.

In respect of the fact that the peristaltic pump and, thus, also the rocker 41 is used in the medical field, and because the mechanical stress on the rocker is relatively high due to the forces to be exerted upon the pump segment via the pressure roller, according to the innovation, the rocker 41 is made from high-strength stainless steel with high chemical resistance and is produced by the metal injection molding (MIM) method. The material used can be, for example, a heat-resistant austenitic chromium-nickel steel (X15CrNiSi25-21), as it is known under the designation 1.4841. Said steel is particularly resistant to oxidation, and it has turned out that the surface qualities which can be achieved by the MIM method are sufficient to make the functional surfaces F1 and F2 sufficiently smooth without any further reworking for pressing the pump segment PS gently and without abrasion into the pump bed and aligning it centrically to the pressure rollers.

The fact that the guide bodies 70, 72 are arranged in one piece with or integrally on the rocker 41, namely each on a swivel bearing eye 50, 52, results in a relatively complex shape of the rocker 41 visible from the representations which can be produced at minimum material cost by the MIM method. At the same time, material can be omitted where it is not important to the function. In this way, it is possible, on the one hand, to design the rocker 41—as illustrated in the view according to FIG. 5—with plenty of material spaces or gaps and, on the other hand, to reinforce the sections where an increased mechanical strength is required. Reinforced bearing eyes 50, 52 can thus be utilized to make use of the braces 76 at the rocker 41 for supporting the otherwise quite delicate guide bodies 70, 72. Simultaneously, the clearance between the swivel bearing eyes 50, 52 can be enlarged to axially encompass a more massive hinge pin holding block 39 (see FIG. 3) of the rotor base body 40 between the two swivel bearing eyes. Also, the bracket arms 58, 60 connecting the swivel bearing eyes 50, 52 to the abutment surface 54 are designed to save material so as to provide plenty of space for receiving the pressure roller. The bearing positions 62, 64 are reinforced so that the pivot shaft 66 can be connected to the bracket arms 58, 60 to prevent twisting and displacement.

In the manufacturing method, the injection-molded blank from the mixture of metal powder and binder can already exhibit all geometric features of the finished rocker 41. These geometric features are not lost either during removal of the usually organic binder or during sintering, for the shrinkage occurring during sintering regularly takes place linearly to final densities of up to 96% so that also the surface of the rocker with sufficient quality is good enough after sintering to perform the above-mentioned functions of the rocker while the pump segment is appropriately protected. For the rest, the correct allowance factor can be exactly determined by test sintering processes.

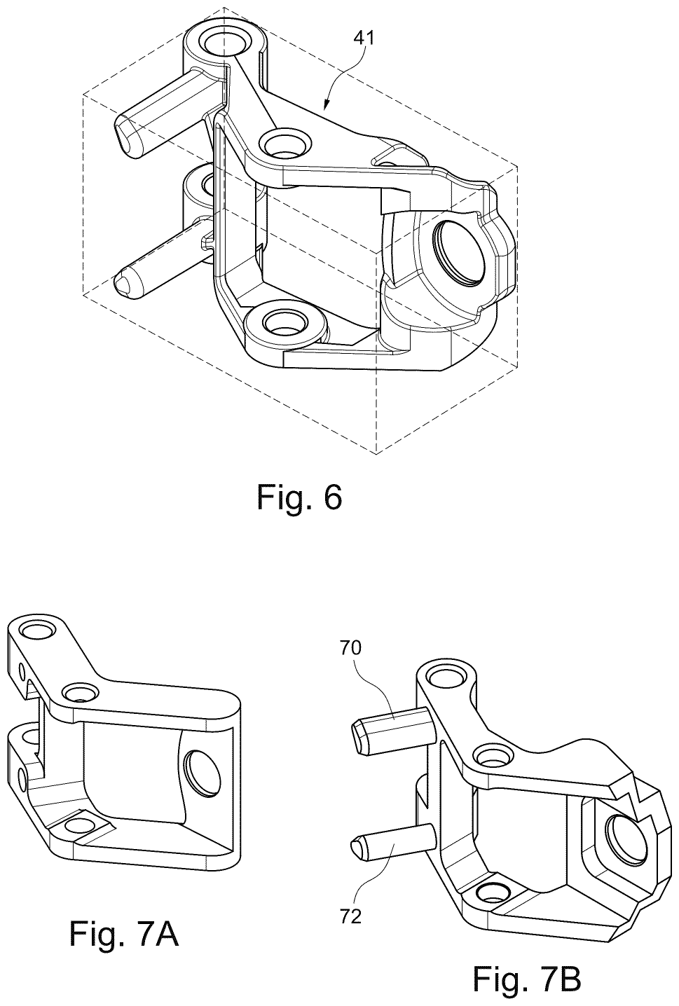

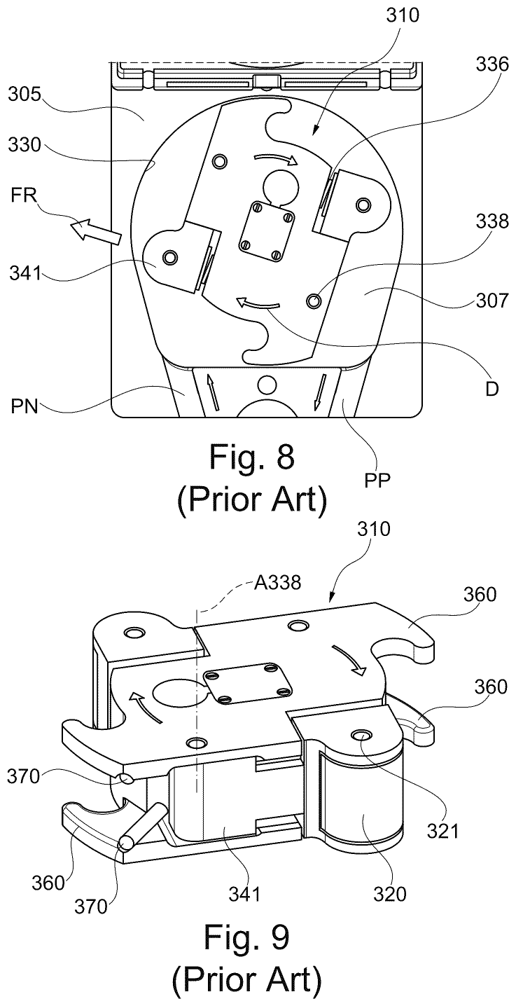

FIG. 6 illustrates which material saving can be achieved by the rocker according to the innovation as compared to a rocker produced by machining. For a metal-cutting machining process, a metal block of about 40 cm³-shown by a broken line—is required about 36 cm³ of material of which would have to be removed in complex working steps. Accordingly, the material expenditure of the above-described rocker amounts to only about 4 cm³ so that considerable saving of raw material is possible.

It is clear from FIG. 7A how a rocker that can be machined at reasonable manufacturing costs differs from the rocker manufactured and configured according to the innovation. It is obvious that only a base body having quite simple surfaces without bracings can be manufactured to which then the components for the insertion aid and the tube guide elements have to be connected. Bracings cannot be materialized at reasonable costs. Only a material which can be easily machined is taken into account as material so that an additional expenditure for a surface treatment is required.

The representation according to FIG. 7B illustrates which additional material expenditure is necessary, if by means of a conventional milling process a rocker is manufactured in which tube guide elements in the form of pin-like guide bodies 70, 72 are integrated in one piece into the rocker.

As compared to this, the rocker according to the innovation—as shown in FIG. 6—is a component of high-strength despite little use of material, wherein further treatment of the surfaces can be omitted.

As a matter of course, deviations from the described embodiment are possible without leaving the basic idea of the present disclosure.

The guide bodies 70, 72 can also be additionally coated, if needed, with a material having abrasion-minimizing sliding properties.

For the manufacture of the rocker 41 also a different powder injection molding method can be applied, such as ceramic powder injection molding.

It is also possible to design the guide bodies 70, 72 as separate components which are then connected to the rocker 41 to form an integral unit.

In the above-described peristaltic pump, two squeeze elements are provided which are arranged at a position of 180° rigidly or at a fixed angle of 180° relative to each other. However, there can also be provided more than two squeeze elements and the squeeze elements can be designed to be positioned at an adjustable angle relative to each other in the direction of rotation. To this end, the squeeze elements can be arranged on rotor arms which may be pivotable in the circumferential direction vis-à-vis the rotor.

The squeeze elements do not have to be designed as squeeze rollers or pressure rollers which advantageously roll off the fluid line in a material-friendly manner. Also, slide shoes which move slidingly over the tube segment may be provided.

Consequently, the present disclosure provides a peristaltic pump, specifically for pumping fluid in an apparatus for extracorporeal blood treatment, comprising a pump housing in which a rotor rotatable about a rotor axis and having at least two squeeze elements offset against each other in the circumferential direction is accommodated and which includes a support surface extending in curved shape around the rotor axis and being radially spaced apart from the rotor, said support surface being arranged to support a tube segment adapted to be radially inserted between the rotor and the support surface. The rotor supports the squeeze element via a rocker which is fixed to the rotor in a pivotable and spring-loaded or cushioned manner. The rocker in turn supports an insertion aid by which the tube segment can be pressed into the pump bed while it is threaded in, and tube guide elements by which the tube segment can be aligned centrally to the squeeze element. In order to provide a peristaltic pump which excels by simplified manufacture while its functionality and service life are increased, the rocker is made from high-strength stainless steel with high chemical resistance and is manufactured by the powder injection molding method, preferably metal injection molding (MIM) method.

### LIST OF REFERENCE SYMBOLS

A rotor axis
D direction of rotation
PN low-pressure connection
PP high-pressure connection
PS pump segment
ES plane of symmetry
F1 functional surface
F2 functional surface
FR radial direction
5 pump housing
7 pump bed
10 rotor
20 squeeze element
30 support surface
38 hinge pin
39 hinge pin holding block
40 rotor base body
42 rotor cover
44 push button
46 guide surface
48 guide surface
50 bearing eye
52 bearing eye
54 abutment surface
56 compression spring
58 upper bracket arm
60 lower bracket arm
62 upper bearing position
64 lower bearing position
66 pivot shaft
70 upper guide body
72 lower guide body
76 brace
78 clearance
305 housing
307 pump bed
310 rotor
320 squeeze elements
321 roller axle
330 support surface
341 rocker
336 compression spring
338 hinge pin
A338 axis
360 guide projections, hooks
370 guide pins

1. A peristaltic pump for pumping fluid in an apparatus for extracorporeal blood treatment, the peristaltic pump comprising:
a pump bed;
a pump housing accommodating a rotor inside the pump housing, the rotor being rotatable about a rotor axis and having a squeeze element, the pump housing having a support surface extending in curved shape around the rotor axis and being radially spaced apart from the rotor, said support surface being arranged to support a tube segment inserted radially between the rotor and the support surface,
the rotor supporting a rocker for the squeeze element, the rocker being mounted on the rotor in a pivotable and spring-loaded or cushioned manner,
the peristaltic pump comprising an insertion aid by which the tube segment, when threaded in, is pressable into the pump bed,
the peristaltic pump further comprising tube guide elements by which the tube segment is alignable centrally to the squeeze element,
the rocker being made from high-strength stainless steel with high chemical resistance and manufactured by a powder injection molding method.

2. The peristaltic pump according to claim 1, wherein functional surfaces of the rocker are not subjected to surface-smoothing treatment after sintering.

3. The peristaltic pump according to claim 1, wherein both the insertion aid and the tube guide elements are integrated in the rocker adjacent to the squeeze element in a direction of rotation of the rotor.

4. The peristaltic pump according to claim 1, wherein the rocker is arranged on the rotor by a swivel bearing arranged offset against the squeeze element in a direction of rotation, the insertion aid and the tube guide elements being fitted integrally on the swivel bearing.

5. The peristaltic pump according to claim 4, wherein the insertion aid is formed by a guide body that extends from the swivel bearing substantially in a radial direction away from the rotor axis and which, as seen in cross-section, substantially takes a rounded wedge shape facing the direction of rotation that is inclined relative to the pump bed at an acute angle.

6. The peristaltic pump according to claim 5, wherein a cross-section of the guide body has a central axis.

7. The peristaltic pump according to claim 5, wherein the guide body comprises:
an inner functional surface that leads in the direction of rotation of the rotor and forms a rounded wedge surface remote from a central plane; and
an outer functional surface that faces the central plane and is formed by a cylindrical segment surface.

8. The peristaltic pump according to claim 7, further comprising a second guide body associated with the guide body and comprising a second cylindrical segment surface, the second guide body designed and oriented symmetrically with respect to the guide body relative to a horizontal plane in parallel to the pump bed so that the cylindrical segment surface and the second cylindrical segment surface facing each other form functional surfaces of the tube guide elements.

9. The peristaltic pump according to claim 8, wherein the horizontal plane is a plane of symmetry of the rocker.

10. The peristaltic pump according to claim 8, wherein the guide body is mounted integrally on a swivel bearing eye of the rocker.

11. The peristaltic pump according to claim 10, wherein the rotor comprises a rotor base body, wherein the second guide body is mounted on a second swivel bearing eye, and wherein the swivel bearing eye and the second swivel bearing eye axially encompass a hinge pin holding block of the rotor base body.

12. The peristaltic pump according to claim 10, wherein the rocker has an end section remote from the swivel bearing eye and forms an abutment surface for a compression spring at the end section, wherein the squeeze element is arranged between the swivel bearing eye and the abutment surface.

13. The peristaltic pump according to claim 1, wherein at least the insertion aid and/or the tube guide elements is/are formed by and/or coated with a material that has abrasion-minimizing sliding properties.
